(19) United States
(12) Patent Application Publication
(10) Pub. No.: US 2004/0072905 A1
(43) Pub. Date: Apr. 15, 2004

(54) Use of nitric oxide synthase inhibitors for the treatment of diabetes

(75) Inventors: Nicholas Charles Turner, London (GB); Valerie Piercy, Potton (GB)

Correspondence Address: GLAXOSMITHKLINE, Corporate Intellectual Property - UW2220, P.O. Box 1539, King of Prussia, PA, 19406-0939, US

(73) Assignee: SmithKline Beecham p.l.c.

(21) Appl. No.: 10/631,051

(22) Filed: Jul. 24, 2003

Related U.S. Application Data
(63) Continuation of application No. 10/290,133, filed on Nov. 7, 2002, now abandoned.
Application No. 10/290,133 is a continuation of application No. 09/850,655, filed on May 7, 2001, now abandoned.
Application No. 09/850,655 is a continuation of application No. 09/367,295, filed on Oct. 7, 1999, now abandoned.
Application No. 09/367,295 is a 371 of international application No. PCT/EP97/00691, filed on Feb. 13, 1997.

(51) Int. Cl. A61K 31/198

(52) U.S. Cl. 514/565

(57) ABSTRACT

A method for the treatment and/or prophylaxis of Type II diabetes, which method comprises the administration, to a human or non-human mammal, of an effective non-toxic pharmaceutically acceptable amount of an NO synthase inhibitor, such as aminoguanidine, or a pharmaceutically acceptable derivative thereof.

This invention relates to a novel method for the treatment of and/or prophylaxis of on-insulin dependant (NIDDM or Type II) diabetes, and in particular to the use of an NO synthase inhibitor, such as aminoguanidine, for the said treatment and/or prophylaxis.

Hydrazinecarboximidamide (hereinafter aminoguanidine) is a known compound (Journal of American Chemical Society, 57, 2730, (1935).

Aminoguanidine is known to be an inhibitor of protein glycation and is under valuation in animal models for the treatment of diabetic complications (Diabetes 42, 21-232 1993; Diabetologia 35, 946-950)

Aminoguanidine is known to be an NO synthase inhibitor (Eur. J Pharmacol., 233, 119-125) and to be useful for the treatment of disease states characterised by over production of NO (European J. of Pharmacology, 233, (1993), 119-125). The inhibition of nitric oxide formation is particularly considered to be linked to the known activity of aminoguanidine in the treatment of diabetic complications European Patent Application, publication number. Aminoguanidine is under evaluation in animal models for the treatment of diabetic complications (Diabetes 42:221-232 1993; Diabetologia 35:946-950).

To date there has been no indication that aminoguanidine or any other NO synthase inhibitor would have a beneficial effect on Type II diabetes itself. As indicated above the emphasis has been focused upon the complications of diabetes. We have now surprisingly discovered that aminoguanidine shows potential for use in the treatment and/or prophylaxis of Type II diabetes per se. In particular, aminoguanidine is indicated to delay or prevent the progression of non-insulin dependent diabetes from hyperinslinaemia to overt diabetes. This novel and surprising effect is considered to be due to the NO synthase inhibitor activity of aminoguanidine on the Type II diabetic pancreas.

Accordingly, the present invention provides a method for the treatment and/or prophylaxis of Type II diabetes, which method comprises the administration, to a human or non-human mammal, of an effective non-toxic pharmaceutically acceptable amount of an NO synthase inhibitor or a pharmaceutically acceptable derivative thereof.

Preferably, the invention provides a method for the prophylactic treatment of Type II diabetes, in particular delaying or preventing the progression from hyperinsulinaemia to hyperglycaemia.

Suitable, inhibitors of NO synthase include proteins and non-protein compounds, such as aminoguanidine, n-monomethylarginine, or other analogues of 1-arginine.

A particular NO synthase inhibitor is aminoguanidine.

When used herein the term ‘NO synthase inhibitor’ refers to an agent that inhibits the formation of nitric oxide from 1-arginine.

The NO synthase inhibitor activity of a compound is assessed in conventional tests such as inhibition of [³H] 1-arginine to [³H] to citrulline or inhibition of nitric oxide generation by cells or tissue extracts or by recombinant nitric oxide synthase isoenzymes in vitro (FASEB.J 1993;7:349-360.

A suitable pharmaceutically acceptable derivative is a pharmaceutically acceptable salt, or a pharmaceutically acceptable solvate thereof.

Suitable pharmaceutically acceptable salts include acid addition salts.

Suitable acid addition salts include pharmaceutically acceptable inorganic salts such as the sulphate, nitrate, phosphate, borate, hydrochloride and hydrobromide and pharmaceutically acceptable organic acid addition salts such as acetate, tartrate, maleate, citrate, succinate, benzoate, ascorbate, methane-sulphonate, a-keto glutarate and a-glycerophosphate, especially the maleate salt.

Suitable pharmaceutically acceptable solvates include hydrates.

The NO synthase inhibitors used in the method of the invention may be prepared according to conventional methods such as the methods disclosed in the above mentioned publications for example aminoguanidine may be prepared according to the methods disclosed in J. Amer. Chem. Soc. 57,2730, (1935).

Salts and/or solvates may be prepared and isolated according to conventional procedures.

In a further aspect the present invention also provides an NO synthase inhibitor, such as aminoguanidine, or a pharmaceutically acceptable derivative thereof, for use in the treatment of and/or prophylaxis of Type II diabetes.

There is also provided an NO synthase inhibitor, such as aminoguanidine, or a pharmaceutically acceptable derivative thereof, for use in the manufacture of a medicament for the treatment and/or prophylaxis of Type II diabetes.

In the above mentioned treatments and/or prophylaxis, the NO synthase inhibitor, such as aminoguanidine, or a pharmaceutically acceptable derivative thereof may be administered per se or preferably as a pharmaceutical composition also comprising a pharmaceutically acceptable carrier.

Accordingly, the present invention also provides a pharmaceutical composition for the treatment and/or prophylaxis of Type II diabetes, which composition comprises an NO synthase inhibitor such as aminoguanidine, or a pharmaceutically acceptable derivative thereof, and a pharmaceutically acceptable carrier therefor.

As used herein the term ‘pharmaceutically acceptable’ embraces compounds, compositions and ingredients for both human and veterinary use: for example the term ‘pharmaceutically acceptable salt’ embraces a veterinarily acceptable salt.

The composition may, if desired, be in the form of a pack accompanied by written or printed instructions for use.

Usually the pharmaceutical compositions of the present invention will be adapted for oral administration, although compositions for administration by other routes, such as by injection and percutaneous absorption are also envisaged.

Particularly suitable compositions for oral administration are unit dosage forms such as tablets and capsules. Other fixed unit dosage forms, such as powders presented in sachets, may also be used.

In accordance with conventional pharmaceutical practice the carrier may comprise a diluent, filler, disintegrant, wetting agent, lubricant, colourant, flavourant or other conventional adjuvant.

Typical carriers include, for example, microcrystalline cellulose, starch, sodium starch glycollate, polyvinylpyrrolidone, polyvinylpolypyrrolidone, magnesium stearate, sodium lauryl sulphate or sucrose.

Most suitably the composition will be formulated in unit dose form. Such unit dose will normally contain an amount of the active ingredient in the range of from 0.1 to 1000 mg, more usually 0.1 to 500 mg, and more especially 0.1 to 250 mg.

Conveniently, the active ingredient may be administered as a pharmaceutical composition hereinbefore defined, and this forms a particular aspect of the present invention.

In the above mentioned treatments the NO synthase inhibitor such as aminoguanidine, or a pharmaceutically acceptable derivative thereof, may be taken in doses such as those described above, one to six times a day in a manner such that the total daily dose for a 70 kg adult will generally be in the range of from 0.1 to 6000 mg, and more usually about 1 to 1500 mg, generally about 0.5 to 10 mg. That is in the range of from 1.429×10⁻³ to 85.714 mg/kg/day, more usually about 1.429×10⁻² to 21.429 mg/kg/day, generally about 7.143×10⁻³ to 0.1429 mg/kg/day.

No unacceptable toxicological effects are observed when active compounds are administered in accordance with the above mentioned invention.

The following Example illustrates the invention but does not limit it in any way.

### EXAMPLE

Methodology of dbdb Mouse Model

The obese db/db mouse is a genetic model of type 2 non-insulin dependent diabetes which is both insulin resistant and hyperglycaemic. Male animals were obtained at 6 weeks of age. Blood samples were taken by tail tip snip for measurement of pre-treatment blood glucose. Animals were allocated into treated and control groups such that the mean and standard deviation of the fasting blood glucose concentrations of each group was similar.

On day 0 of the study a group of obese animals and their lean litter mates were killed for measurement of baseline biochemistry and histology. In addition one group of animals (control; n=14) were fed a standard diet and a further group received aminoguanidine (500 mg/kg; n=14) in the same diet. Animals were allowed free access to food and water and their intake measured daily. At weekly intervals 24 hr urine output was also measured. Mice (n=7) were killed at 30 days and 85 days from commencement of treatment. Blood was taken for measurement of glucose and insulin concentrations and the pancreas removed for histological analysis and for measurement of pancreatic insulin.

Data From dbdb Mouse Model

Food intake and body weight gain of the control and treated groups was similar throughout the experimental period.

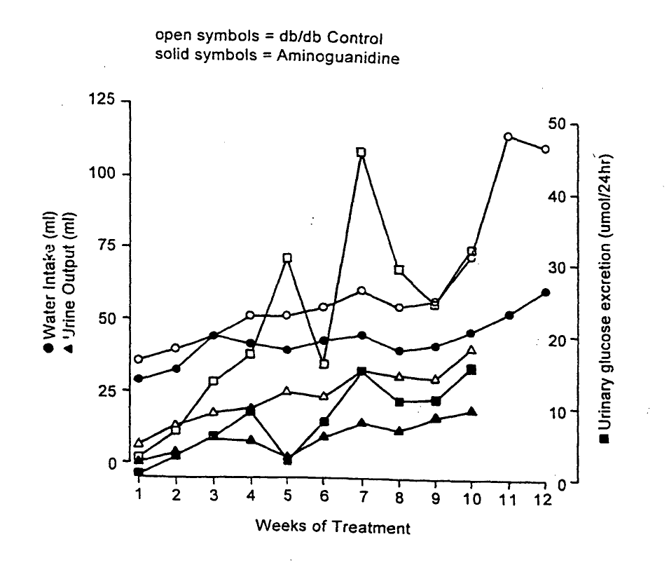

Immediately prior to dosing obese animals were normoglycaemic (blood glucose 10.4±0.97 mM) but were hyperinsulinaemic compared to their lean litter mates (serum isulin 127±37 ng/ml in obese animals 3.05±1.03 ng/ml in leans). By day 30 of the dosing period the obese control group were hyperglycaemic (blood glucose 24.9±1.0 mM) and had markedly lower serum insulin levels (30.75±4.3 mM) compared to the pre-treatment values. By day 85 of the treatment period, fasting blood glucose had risen to 28.1±2 mM and serum insulin concentrations had fallen further, to 11.7±1.8 ng/ml. Aminoguanidine attenuated the fall in fasting insulin concentrations (58.3±13 ng/ml on day 30, 23.3±4.1 ng/ml on day 85) and on day 85 had significantly reduced the prevailing fasting hyperglycaemia (21±1.7 mM). Pancreatic insulin content of the aminoguanidine treated group of obese animals was twice that of the untreated animals (64.3±17.8 ng/mg pancreas compared to 30.0±2.6 ng/mg, respectively). From day 63 of the experimental period obese control animals were markedly polydypsic and polyuric compared to day 7. This increase in water intake and urine output is a characteristic of diabetes (hyperglycaemia) and was prevented by treatment with aminoguanidine (FIG. 1). Similarly urinary glucose excretion increased steadily over the experimental period, in both untreated and treated animals, but from day 35 was lower in the aminoguanidine treated group (FIG. 1). The development of diabetes (hyperglycaemia) was associated with changes in islet morphology, and the islets of untreated control animals were markedly hypertrophic, disorganised and had irregular boundaries. Islet insulin content was markedly depleted. On day 30 of the treatment period normal islet morphology was preserved in the aminoguanidine treated animals, and islet insulin content was greater.

1. A method for the treatment and/or prophylaxis of Type II diabetes, which method comprises the administration, to a human or non-human mammal, of an effective non-toxic pharmaceutically acceptable amount of an NO synthase inhibitor or a pharmaceutically acceptable derivative thereof.

2. A method according to claim 1, for the prophylactic treatment of Type II diabetes.

3. A method according to claim 1 or claim 2, for delaying or preventing the progression from hyperinsulinaemia to hyperglycaemia.

4. A method according to any one of claims 1 to 3, wherein the NO synthase inhibitor is selected from aminoguanidine, n-monomethylarginine, or other analogues of 1-arginine.

5. A method according to any one of claims 1 to 4, wherein the NO synthase inhibitor is aminoguanidine.

6. An NO synthase inhibitor, or a pharmaceutically acceptable derivative thereof, for use in the treatment of and/or prophylaxis of Type II diabetes.

7. An NO synthase inhibitor, or a pharmaceutically acceptable derivative thereof, for use in the manufacture of a medicament for the treatment and/or prophylaxis of Type II diabetes.

8. A pharmaceutical composition for the treatment and/or prophylaxis of Type II diabetes, which composition comprises an NO synthase inhibito, or a pharmaceutically acceptable derivative thereof, and a pharmaceutically acceptable carrier therefor.
